(19) United States
(12) Patent Application Publication
(10) Pub. No.: US 2013/0266643 A1
(43) Pub. Date: Oct. 10, 2013

(54) Compositions comprising glyceryl-triacetate (GTA) and uses in human performance optimization and thermogenesis

(71) Applicant: HPO LIFE SCIENCES, INC., Cos Cob, CT (US)

(72) Inventor: John R Moffett, North Potomac, MD (US)

(21) Appl. No.: 13/858,492

(22) Filed: Apr. 8, 2013

Related U.S. Application Data
(60) Provisional application No. 61/686,687, filed on Apr. 10, 2012.

(51) Int. Cl. A61K 31/225; A61K 45/06

(52) U.S. Cl. CPC A61K 31/225; A61K 45/06
USPC 424/451; 514/547; 424/490

(57) ABSTRACT

Described herein are compositions comprising glyceryl-triacetate (GTA) for use in delivering non-glucose energy deriving metabolites during physical performance, which reduce lactate production and serve as thermogenic agents.

### RELATED APPLICATION DATA

This application claims the priority of prior U.S. provisional application Ser. No. 61/686,687 filed on Apr. 10, 2012, which is hereby incorporated by reference herein in its entirety.

### TECHNICAL FIELD

Compositions of the present invention relate generally to compositions for providing energy, and more specifically to compositions for use in delivering non-glucose energy deriving metabolites during physical performance.

### BACKGROUND ART

Most energy in the body is derived from blood glucose. Sugar utilization requires insulin to allow entry of glucose into cells of the body. The multi-step process of glycolysis converts the cellular glucose into the basic metabolite pyruvate (aka pyruvic acid). Pyruvate is then converted to acetyl coenzyme A (acetyl CoA) which is the primary molecule that goes on to be oxidized for energy production in the form of adenosine triphosphate or ATP. When pyruvate is produced in muscles during vigorous exercise, but insufficient oxygen is delivered to generate ATP through oxidative phosphorylation, some of the pyruvate is converted to lactate, which causes lactic acidosis, acidification of muscle and muscle fatigue as levels in muscle rise.

After blood glucose is used up during vigorous exercise, stored liver and muscle glycogen must be metabolized for continued muscle activity. Glycogen is broken down directly to glucose, which can then be used for continued ATP production through glycolysis. During prolonged physical activity, when glucose levels in the blood are low and muscle and liver glycogen stores are depleted, the liver, muscles, kidneys, adipose tissue and other organs in the body convert stored fats (e/g., triglycerides) into acetyl CoA via the process known as beta-oxidation, which then continues to fuel cellular energy production until more nutrients can be derived from a meal. Ketone bodies (acetoacetate, beta-hydroxybutyrate and acetone) are also produced in organs such as the liver, and released to the circulation to continue fueling physical activity after glucose and glycogen are depleted. Ketone bodies can be readily converted into energy (ATP) in all cells of the body including muscle cells.

It is less well known that acetate is also released along with ketone bodies from the liver under ketogenic (fat burning) conditions (Seufert et al., 1974; Leighton et al., 1989). It is also not generally known that muscle cells are among the most adept at utilizing blood acetate for energy production.

Currently, modern energy drinks and energy bars provide caloric energy in the form of sugars, with the addition of mild stimulants, such as caffeine. Energy enhancing products that use other metabolic sources to provide energy more rapidly than sugar-based products and target the calories to skeletal muscles would be more desirable. In addition, by reducing the use of sugars as a source of muscle energy, pyruvate production is limited, thus reducing lactic acidosis during prolonged exertion and exercise.

### SUMMARY OF THE EMBODIMENTS

Glyceryl-triacetate (GTA) provides a rapid source of acetyl CoA synthesis for prolonged aerobic ATP regeneration. Accordingly, it is an object of the present invention to provide an immediate metabolic energy source in a liquid drink, energy bar, capsule, food product or transdermal patch containing GTA as a calorie source for increasing skeletal muscle metabolic energy levels during physical performance (e.g., medical, military and sports applications). Moreover, if glucose consumption is replaced with GTA prior to such physical performance, less lactate is produced in muscles, lactic acidosis is reduced and muscle fatigue is delayed and/or decreased, allowing for prolonged exertion.

In one aspect, the invention provides a method for increasing physical performance of a human subject, said method comprising administering a liquid formula comprising about 0.5% v/v to about 5% v/v glyceryl-triacetate to the human subject, thereby increasing physical performance in the human subject, wherein the increase in physical performance comprises increased ATP production from acetyl CoA. In other embodiments, the increase in physical performance comprises an increase in blood oxygen content or carbon dioxide output or a decrease in blood lactate levels.

In another aspect, the invention provides a method for increasing energy production in the heart and skeletal muscle of a human subject, said method comprising administering a liquid formula comprising about 0.5% v/v to about 5% v/v glyceryl-triacetate (GTA) to the human subject, thereby increasing energy production in the in the heart and skeletal muscle of the human subject.

In one embodiment, increasing energy production comprises increasing ATP production from acetyl CoA.

In another embodiment, the invention provides a method for increasing energy production in the heart and skeletal muscle of a human subject, said method comprising administering a food product comprising about 0.5% to about 10% w/w glyceryl-triacetate to the human subject, thereby increasing energy production in the in the heart and skeletal muscle of the human subject. In specific embodiments, the human subject has about 99% to about 85% blood oxygen content, and/or a blood lactate level of about 2 millimolar to about 20 millimolar.

In another aspect, the invention provides a method of decreasing, delaying or preventing lactic acidosis in a human subject, said method comprising administering a liquid formula comprising about 0.5% v/v to about 5% v/v glyceryl-triacetate, or a food product comprising about 0.5% to about 10% w/w glyceryl-triacetate to the human subject in an amount and duration sufficient to increase ATP production from acetyl CoA, thereby decreasing, delaying or preventing lactic acidosis in the human subject.

In one embodiment, the liquid formula or food product further comprises anti-esterase flavonoids.

In another embodiment, the anti-esterase flavonoid is selected from the group consisting of, but not limited to, naringenin, morin, galangin, quercetin, kaempferol and ursolic acid and combinations thereof.

In yet another embodiment, the liquid formula or food product comprises a supplement selected from the group consisting of, but not limited to, L-carnitine, vitamins, amino acids, creatine and taurine and combinations thereof

In yet another embodiment, the liquid formula or food product comprises a sugar selected from the group consisting of sucrose, glucose, fructose, ribose and maltose and combinations thereof

In yet another embodiment, the liquid formula or food product comprises a stimulant selected from the group consisting of caffeine, theobromine and theophylline and combinations thereof.

In yet another embodiment, the glyceryl-triacetate is microencapsulated. The glyceryl-triacetate can be microencapsulated with an anti-esterase flavonoid.

In yet another embodiment, the glyceryl-triacetate is contained in a capsule, pill or soft gel cap. The capsule, pill or soft gel cap can further comprise an anti-esterase flavonoid.

In yet another embodiment, the glyceryl-triacetate is contained in a transdermal patch.

In yet another embodiment, the liquid formula or food product is orally administered.

In yet another aspect, the invention provides a method for increasing brown adipose tissue metabolism and thermogenesis in a human subject, said method comprising administering a liquid formula comprising about 0.5% v/v to about 5% v/v glyceryl-triacetate or a food product comprising about 0.5% to about 10% w/w glyceryl-triacetate to the subject, thereby increasing brown adipose tissue metabolism and thermogenesis in the human subject.

In one embodiment, increasing brown adipose tissue metabolism and thermogenesis increases the body temperature of the human subject by about 0.1° F. to about 2° F.

Other aspects, embodiments and features of the invention will become apparent from the following detailed description of the invention when considered in conjunction with the accompanying figures. The accompanying figures are for schematic purposes and are not intended to be drawn to scale. In the figures, each identical or substantially similar component that is illustrated in various figures is represented by a single numeral or notation. For purposes of clarity, not every component is labeled in every figure. Nor is every component of each embodiment of the invention shown where illustration is not necessary to allow those of ordinary skill in the art to understand the invention.

### BRIEF DESCRIPTION OF THE DRAWINGS

The preceding summary, as well as the following detailed description of the invention, will be better understood when read in conjunction with the attached drawings. For the purpose of illustrating the invention, presently preferred embodiments are shown in the drawings. It should be understood, however, that the invention is not limited to the precise arrangements and instrumentalities shown.

FIG. 1 shows a diagrammatic representation of glyceryl-triacetate (GTA). GTA is a simple triglyceride with 3 acetate molecules bound to a glycerol molecule through three ester linkages (O).

FIG. 2 illustrates in schematic the biochemical mechanism by which GTA acts to provide a rapid source of energy to muscle cells in the form of acetyl coenzyme A (acetyl CoA) which can then enter the citric acid cycle in mitochondria for ATP production. As shown, ACS2 is acetyl coenzyme A synthase-2, CoSH is coenzyme A, and GTA is glyceryl-triacetate. GTA is acted on in muscle cells by esterase or lipase enzymes which convert the GTA into glycerol and three acetate molecules. The acetate is then acted upon by the enzyme acetyl coenzyme A synthase-2 (present in the mitochondria of muscle cells) to produce acetyl CoA. Acetyl CoA is the primary source of ATP production via the citric acid cycle in muscle cells.

FIG. 3 illustrates in schematic form the common metabolic routes to acetyl CoA synthesis in muscle cells. The three ATP regeneration systems include; 1) the Phosphagen system (very fast ATP regeneration, but very low capacity), 2) Glycolysis (fast ATP regeneration, but low capacity) and 3) Aerobic ATP regeneration (slow but very high capacity). As shown, ATP is adenosine triphosphate, ADP is adenosine diphosphate, P is phosphate, and TCA cycle is the tricarboxylic acid cycle (aka citric acid cycle). Lipids undergo beta oxidation to form acetyl CoA, some amino acids can be catabolized to acetyl CoA, and carbohydrates undergo glycolysis to produce pyruvate, which can be converted into acetyl CoA. GTA provides a direct route to acetyl CoA in muscle cells as compared with lipids, amino acids or carbohydrates due to the fewer number of enzymatic steps (only 2), and does not involve the production of pyruvate or lactate.

### DETAILED DESCRIPTION OF SPECIFIC EMBODIMENTS

Definitions. As used in this description and the accompanying claims, the following terms shall have the meanings indicated, unless the context otherwise requires.

By “an effective amount” is meant the amount of a required agent or composition comprising GTA to ameliorate the symptoms of a disorder (e.g., lactic acidosis, muscle fatigue) to an untreated subject. The effective amount of GTA used to practice the present invention for varies depending upon the manner of administration, the age, body weight, and general health of the subject.

By “increasing physical performance” is meant an increase in blood oxygen content, carbon dioxide output, ATP production, a decrease in blood lactate levels (Poulos et al., 2011), or prolonged exercise capacity. Prolonged exercise capacity can be determined, for example, by monitoring endurance in a subject administered GTA or placebo, and/or monitoring endurance in a subject administered equal caloric amounts of sugar or GTA and in either case, determining that the subject can continue exercise for a longer duration without muscle fatigue and cramping following GTA administration.

By “increasing brown adipose tissue metabolism and thermogenesis” is meant to increase the oxidation of nutrients in brown adipose tissue thus generating increased heat which can be measured by various means, including, but not limited to, whole-room calorimeters, skin temperature monitoring, thermal imaging and, cold tolerance tests conducted in a freezer room.

By “food product” is meant any GTA containing substance comprised of non-liquid (e.g., solid, semi-solid, frozen) edible compositions.

The term “reduced” or “reduce” or “decrease” as used herein generally means a decrease by a statistically significant amount. However, for avoidance of doubt, “reduced” means a decrease by at least 5% as compared to a reference level, for example a decrease by at least about 10%, or at least about 20%, or at least about 30%, or at least about 40%, or at least about 50%, or at least about 60%, or at least about 70%, or at least about 80%, or at least about 90% or up to and including a 100% decrease (i.e. substantially absent or below levels of detection), or any decrease between 5-100% as compared to a reference level, as that term is defined herein.

The term “increase” as used herein generally means an increase by a statistically significant amount. However, for avoidance of doubt, “increase” means an increase by at least 5% as compared to a reference level, for example an increase by at least about 10%, or at least about 20%, or at least about 30%, or at least about 40%, or at least about 50%, or at least about 60%, or at least about 70%, or at least about 80%, or at least about 90% or up to and including a 100% increase (i.e. significantly above levels of detection), or any increase between 10-100% as compared to a reference level, as that term is defined herein.

As used herein, the term “standard” or “reference” refers to a measured physical parameter including but not limited to an amount of blood oxygen content, carbon dioxide output, lactic acid levels, glycogen levels or ATP production in a known sample against which another sample is compared; alternatively, a standard can simply be a reference number that represents an amount of blood oxygen content, carbon dioxide output, lactic acid levels, glycogen levels or ATP production that defines a baseline for comparison. The reference number can be derived from either a sample taken from an individual, or a plurality of individuals. That is, the “standard” does not need to be a sample that is tested, but can be an accepted reference number or value. A series of standards can be developed that take into account an individual's status, e.g., with respect to age, gender, weight, height, ethnic background etc. A standard level can be obtained for example from a known biological sample from a different individual (e.g., not the individual being tested) undergoing physical exercise and in some embodiments, undergoing lactic acidosis. A known sample can also be obtained by pooling samples from a plurality of individuals to produce a standard over an averaged population. Additionally, a standard can be synthesized such that a series of standards are used to quantify the amount of blood oxygen content, carbon dioxide output, lactic acid levels, glycogen levels or ATP production in an individual's sample. A biological sample from the individual to be tested can be obtained at an earlier time point (presumably prior to the onset of physical exercise) and serve as a standard or reference compared to a biological sample taken from the same individual after the onset of physical exercise. In such instances, the standard can provide a measure of the efficacy of GTA. In specific embodiments, the term “standard” or “reference” refers to the degree of metabolism in the brown adipose tissue of an individual which is compared to that of another individual or of a prior reading from the same individual according to the aforementioned methods for sample comparison.

A “subject” is a vertebrate, including any member of the class mammalia, including humans, domestic and farm animals, and zoo, sports or pet animals, such as mouse, rabbit, pig, sheep, goat, cattle and higher primates.

In this disclosure, “comprises,” “comprising,” “containing” and “having” and the like can have the meaning ascribed to them in U.S. Patent law and can mean “includes,” “including,” and the like; “consisting essentially of” or “consists essentially” likewise has the meaning ascribed in U.S. Patent law and the term is open-ended, allowing for the presence of more than that which is recited so long as basic or novel characteristics of that which is recited is not changed by the presence of more than that which is recited, but excludes prior art embodiments.

Other definitions appear in context throughout this disclosure.

### Compositions and Methods of the Invention

During fasting the liver produces and releases some acetate to the blood along with ketone bodies for use as a source of energy. Acetate has not typically been considered a major source of metabolic energy in humans. However, blood acetate is a more direct and immediate source of energy for muscle cells than blood glucose. Acetate is metabolized predominantly by the enzyme acetyl CoA synthase-2 (ACS2), which is present at very high levels in muscle cells and brown adipose tissue cells (Fujino et al., 2001; Sakakibara et al., 2009; Hallows et al., 2006). In addition, when making the critical cellular metabolite acetyl coenzyme A (acetyl CoA) from acetate for energy production, the multi-step process of glycolysis is bypassed.

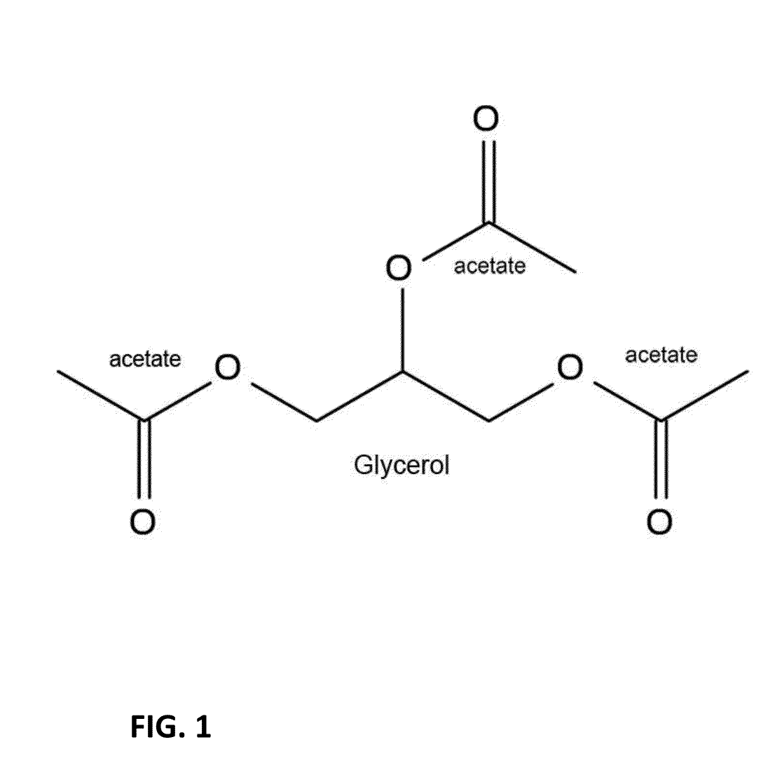

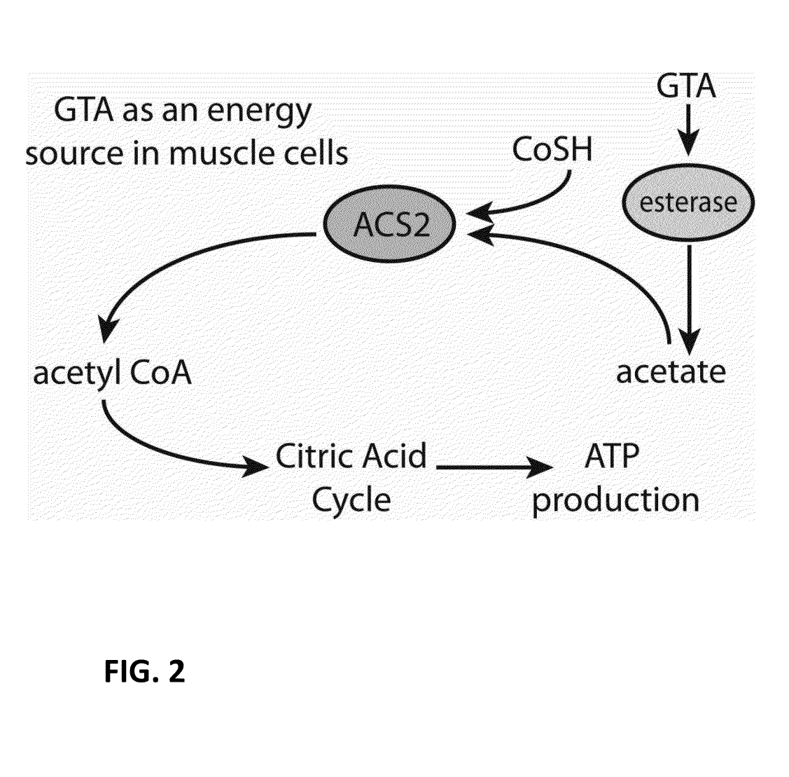

GTA, also known as Triacetin®, is a synthetic, non-toxic triester of 3 acetate molecules on a glycerol molecular backbone as illustrated in FIG. 1. GTA is acted on by esterase and lipase enzymes in the blood and all tissues of the body, and this enzymatic action releases the metabolic payload to muscle cells in the form of acetate. The acetate derived from GTA can then be rapidly converted to acetyl CoA in muscle cells by acetyl CoA synthase-2 as illustrated in FIG. 2. Acetyl CoA is then oxidized for the bioenergetic synthesis of ATP in muscle cells without the buildup of lactate.

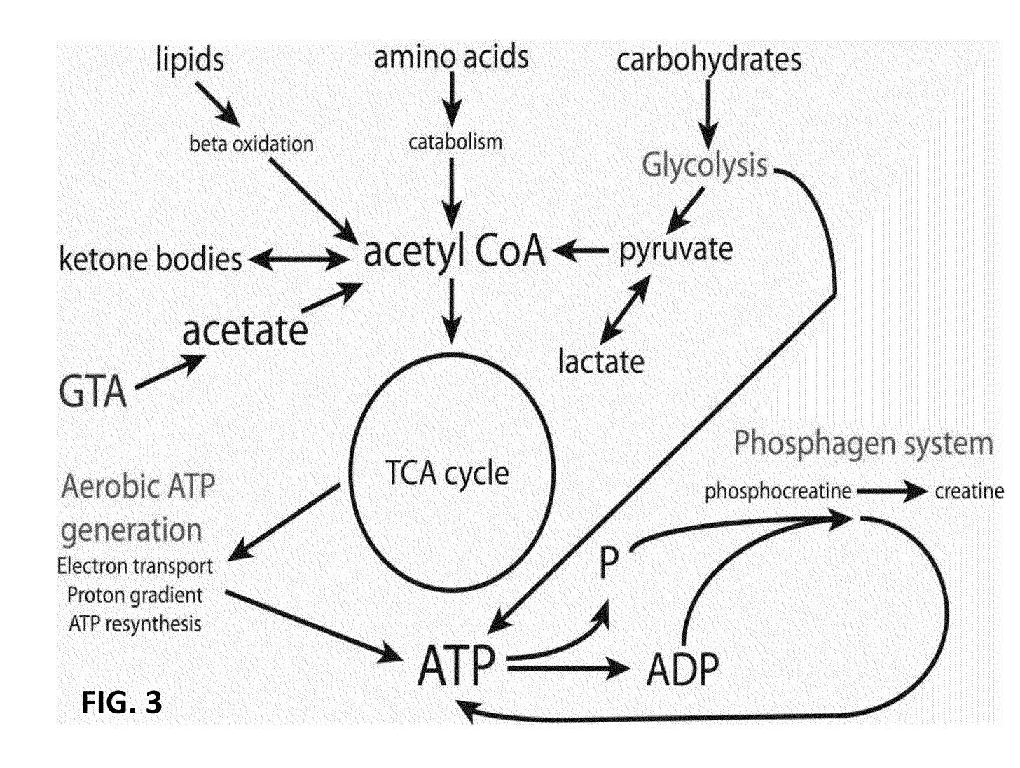

Therefore, GTA is uniquely suited to provide a rapid source of metabolic energy directly to muscle cells for improved or prolonged physical performance. GTA is quickly and efficiently absorbed in the intestine and distributes to tissues rapidly. Accordingly, GTA is converted into acetyl coenzyme A in only two enzymatic steps (esterase or lipase action, followed by acetyl coenzyme A synthase action), as opposed to the ten enzymatic steps required to convert glucose into acetyl CoA via glycolysis and pyruvate production. Therefore, even in the absence of insulin, GTA can deliver metabolic energy to muscle cells much more rapidly and efficiently than sugars, as illustrated in FIG. 3.

Accordingly, compositions and methods of the invention decrease, delay or prevent lactic acidosis. A subject at risk for lactic acidosis, for example, is one having about 99% to about 85% blood oxygen content (e.g., about 99%, 98%, 97%, 96%, 95%, 94%, 93%, 92%, 91%, 90%, 89%, 88%, 87%, 86%, to about 85% blood oxygen content), and/or about 2 millimolar (mM) to about 20 mM blood lactate levels (e.g., about 2, 3, 4, 5, 6, 7, 8, 9, 10, 11, 12, 13, 14, 15, 16, 17, 18, 19, to about 20 mM blood lactate levels) and/or undergoing physical performance.

GTA compositions of the invention can comprise liquid formulations containing about 0.5% to about 5.0% v/v GTA (e.g., about 0.5%, 1.0%, 2.0%, 3.0%, 4.0%, or about 5.0% v/v GTA). In some embodiments, the GTA compositions of the invention are formulated with a pharmaceutically acceptable carrier. In other embodiments the GTA may be formulated with emulsifiers to increase the GTA content up to about 10% (about 6.0%, 7.0%, 8.0%, 9.0%, or about 10.0% v/v GTA). Liquid compositions and formulations of the invention can be administered, topically, orally or by local administration, such as by injection or transdermally via a transdermal patch. In specific embodiments, GTA compositions of the invention can be microencapsulated in order to prolong the release of GTA in the gut.

GTA compositions of the invention can further comprise solid dosage forms for oral ingestion (e.g., food products) containing about 0.5% to about 10.0% w/w GTA (e.g., 0.5%, 1.0%, 2.0%, 3.0%, 4.0%, 5.0%, 6.0%, 7.0%, 8.0%, 9.0% to about 10% w/w GTA).

In specific embodiments, GTA can be used in combination with sugars to provide additional ATP generation via both energy sources in parallel. Such sugars include but are not limited to sucrose, glucose, fructose, maltose and ribose.

In other specific embodiments, GTA can be also used in combination with other energy facilitating metabolites including but not limited to L-carnitine, vitamins, amino acids, creatine and taurine.

In other specific embodiments, the GTA compositions of the invention include anti-esterase flavonoids, such as those found in grapefruit juice (Li et al., 2007), in order to prevent GTA utilization in the intestine and liver, thus delivering more to muscles and heart. Preferably, the anti-esterase flavonoid reduces hydrolysis of GTA in the gut and/or increases acetate delivery to muscle cells. GTA can be combined with anti-esterase compounds including but not limited to naringenin, morin, galangin, quercetin, ursolic acid, and kaempferol to increase delivery of GTA to muscle cells.

GTA compositions of the invention can be used as a diet-induced thermogenic agent for increasing non-exercise thermogenesis in brown adipose tissue. Thermogenesis in mammals in general and humans in particular, involves muscular heat generation associated with shivering, and non-shivering heat production which occurs predominantly in brown adipose tissue. Brown adipose tissue cells contain many mitochondria that contain an uncoupling protein that shunts metabolic energy from ATP production to heat generation. GTA-derived acetate is preferentially converted to heat energy in brown adipose tissue. Compositions comprising GTA would provide a rapid source of energy for muscle and brown fat heat generation enabling longer physical performance in cold conditions. A human subject undergoing physical performance and thereby having about a body temperature of about 37 C or less can increase body temperature by about 0.05° to about 1° C. (e.g., about 0.05°, 0.1°, 0.2°, 0.5°, 1° C.) by consuming GTA compositions of the invention prior to or during physical performance, or in cold environments to prolong exposure times.

It will be understood that the invention may be embodied in other specific forms without departing from the spirit or central characteristics thereof. The present examples and embodiments, therefore, are to be considered in all respects as illustrative and not restrictive, and the invention is not to be limited to the details given herein. The recitation of a listing of elements in any definition of a variable herein includes definitions of that variable as any single element or combination (or subcombination) of listed elements. The recitation of an embodiment herein includes that embodiment as any single embodiment or in combination with any other embodiments or portions thereof.

### INCORPORATION BY REFERENCE

All patents, patent applications and publications mentioned in this specification are herein incorporated by reference to the same extent as if each independent patent and publication was specifically and individually indicated to be incorporated by reference. Incorporation by reference herein includes, but is not limited to: Ariyannur P S, Moffett J R, Madhavarao C N, Arun P, Vishnu N, Jacobowitz D, Hallows W C, Denu J M, Namboodiri A M. 2010. Nuclear-cytoplasmic localization of acetyl coenzyme A synthetase-1 in the rat brain. J Comp Neurol 518:2952-2977. Bailey J W, Barker R L, Karlstad M D. 1992. Total parenteral nutrition with short- and long-chain triglycerides: triacetin improves nitrogen balance in rats. J Nutr 122:1823-1829. Bailey J W, Haymond M W, Miles J M. 1991. Triacetin: a potential parenteral nutrient. JPEN J Parenter Enteral Nutr 15:32-36. Fiume M Z. 2003. Final report on the safety assessment of triacetin. Int J Toxicol 22 Suppl 2:1-10. Fujino T, Kondo J, Ishikawa M, Morikawa K, Yamamoto T T. 2001. Acetyl-CoA synthetase 2, a mitochondrial matrix enzyme involved in the oxidation of acetate. J Biol Chem 276:11420-11426. Goldberg R P, Brunengraber H. 1980. Contributions of cytosolic and mitochondrial acetyl-CoA syntheses to the activation of lipogenic acetate in rat liver. Adv Exp Med Biol 132:413-418. Hallows W C, Lee S, Denu J M. 2006. Sirtuins deacetylate and activate mammalian acetyl-5 CoA synthetases. Proc Natl Acad Sci U S A 103:10230-10235. Ingram-Smith C, Smith K S. 2007. AMP-forming acetyl-CoA synthetases in Archaea show unexpected diversity in substrate utilization. Archaea 2:95-107. Leighton F, Bergseth S, Rortveit T, Christiansen EN, Bremer J. 1989. Free acetate production by rat hepatocytes during peroxisomal fatty acid and dicarboxylic acid oxidation. J Biol Chem 264:10347-10350. Li P, Callery PS, Gan LS, Balani SK. 2007. Esterase inhibition by grapefruit juice flavonoids leading to a new drug interaction. Drug Metab Dispos 35:1203-1208. Luong A, Hannah VC, Brown MS, Goldstein JL. 2000. Molecular characterization of human acetyl-CoA synthetase, an enzyme regulated by sterol regulatory element-binding proteins. J Biol Chem 275:26458-26466. Ogawa T, Moriwaki N, Fujii R, Tanaka K, Mori E, Saitou M, Yoshizawa H, Sakaguchi H. 1992. Triacetin as food additive in gummy candy and other foodstuffs on the market. Kitasato Arch Exp Med 65:33-44. Poulos S P, Rains T M, Rubin M, Kelly K, Maki K C. 2011. Acetate containing sports beverages appear to alter buffering capacity during endurance exercise in trained male athletes. Journal of the International Society of Sports Nutrition 8:10. Sakakibara I, Fujino T, Ishii M, Tanaka T, Shimosawa T, Miura S, Zhang W, Tokutake Y, Yamamoto J, Awano M, Iwasaki S, Motoike T, Okamura M, Inagaki T, Kita K, Ezaki O, Naito M, Kuwaki T, Chohnan S, Yamamoto T T, Hammer R E, Kodama T, Yanagisawa M, Sakai J. 2009. Fasting-induced hypothermia and reduced energy production in mice lacking acetyl-CoA synthetase 2. Cell Metab 9:191-202. Seufert C D, Graf M, Janson G, Kuhn A, Soling H D. 1974. Formation of free acetate by isolated perfused livers from normal, starved and diabetic rats. Biochem Biophys Res Commun 57:901-909. Shapira J, Mandel A D, Quattrone P D, Bell N L. 1969. Current research on regenerative systems. Life Sci Space Res 7:123-129. Wellen K E, Hatzivassiliou G, Sachdeva U M, Bui T V, Cross J R, Thompson C B. 2009. ATP-citrate lyase links cellular metabolism to histone acetylation. Science 324:1076-1080.

What is claimed is:

1. A method for increasing energy production in the heart and skeletal muscle of a human subject, said method comprising administering a liquid formula comprising about 0.5% v/v to about 5% v/v glyceryl-triacetate (GTA) or a food product comprising about 0.5% to about 10% w/w GTA to the human subject having about 99% to about 85% blood oxygen content and/or a blood lactate level of about 2 millimolar to about 20 millimolar, thereby increasing energy production in the heart and skeletal muscle of the human subject.

2. The method of claim 1, wherein increasing energy production comprises increasing ATP production from acetyl CoA.

3. The method of claim 1, wherein the liquid formula or food product further comprises ingredients selected from the group consisting of L-carnitine, ribose, and anti-esterase flavonoid and combinations thereof.

4. The method of claim 3, wherein the anti-esterase flavonoid is selected from the group consisting of naringenin, morin, galangin, quercetin, ursolic acid and kaempferol and combinations thereof.

5. The method of claim 1, wherein the glyceryl-triacetate is microencapsulated.

6. The method of claim 5, wherein the glyceryl-triacetate is microencapsulated with an anti-esterase flavonoid.

7. The method of claim 1, wherein the glyceryl-triacetate is contained in a capsule, pill or soft gel cap.

8. The method of claim 7 wherein the capsule, pill or soft gel cap further comprises an anti-esterase flavonoid.

9. The method of claim 1, wherein the glyceryl-triacetate is contained in a transdermal patch.

10. The method of claim 1, wherein the liquid formula is orally administered.

11. The method of claim 1, wherein the liquid formula or food product comprises ingredients selected from the group consisting of a sugar, stimulant and anti-esterase flavonoid.

12. The method of claim 1, wherein the liquid formula or food product comprises a supplement selected from the group consisting of L-carnitine, vitamins, amino acids, creatine and taurine and combinations thereof

13. The method of claim 1, wherein the liquid formula or food product comprises a sugar selected from the group consisting of sucrose, glucose, fructose, ribose and maltose and combinations thereof.

14. The method of claim 1, wherein the liquid formula or food product comprises a stimulant selected from the group consisting of caffeine, theobromine and theophylline and combinations thereof.

15. A method of decreasing, delaying or preventing lactic acidosis in a human subject, said method comprising administering a liquid formula comprising about 0.5% v/v to about 5% v/v glyceryl-triacetate or a food product comprising about 0.5% to about 10% w/w glyceryl-triacetate to the human subject in an amount and duration sufficient to increase ATP production from acetyl CoA, thereby decreasing, delaying or preventing lactic acidosis in the human subject.

16. A method for increasing brown adipose tissue metabolism and thermogenesis in a human subject, said method comprising administering a liquid formula comprising about 0.5% v/v to about 5% v/v glyceryl-triacetate or a food product comprising about 0.5% to about 10% w/w glyceryl-triacetate to the subject, thereby increasing brown adipose tissue metabolism and thermogenesis in the human subject.

17. The method of claim 16, wherein increasing brown adipose tissue metabolism and thermogenesis increases the body temperature of the human subject by about 0.05° C. to about 1° C.
